(19) United States
(12) Patent Application Publication
(10) Pub. No.: US 2025/0235286 A1
(43) Pub. Date: Jul. 24, 2025

(54) OPTICAL MEASUREMENT SYSTEMS

(71) Applicants: Smith & Nephew, Inc., Memphis, TN (US); Smith & Nephew Orthopaedics AG, Zug (CH)

(72) Inventor: Brett J. Bell, Mendon, UT (US)

(21) Appl. No.: 19/012,289

(22) Filed: Jan. 7, 2025

Related U.S. Application Data
(60) Provisional application No. 63/622,142, filed on Jan. 18, 2024.

(51) Int. Cl. A61B 90/00; G06T 7/00

(52) U.S. Cl. CPC A61B 90/361; A61B 90/06; A61B 90/37; G06T 7/0014; A61B 2090/373; G06T 2207/10021; G06T 2207/20; G06T 2207/30004

(57) ABSTRACT

Examples relate to an intraoperative optical strain measurement systems to determine strain within a target tissue material to receive an implant; the system comprising: an input for receiving temporally consecutive images of the target issue material; the temporally consecutive images comprising: at least one reference image and at least one subsequent image taken after the at least one reference image; a strain measurement processor comprising: measurement calculation circuitry, responsive to a data associated with a difference between the at least one reference image and the at least one subsequent image, to determine strain measurement data indicative of strain within the target tissue material, and an output for outputting strain measurement data associated the measure of strain.

### CROSS-REFERENCE TO RELATED APPLICATIONS

This application claims the benefit of U.S. Provisional Application No. 63/622,142, filed on Jan. 18, 2024. The entire disclosure of the application referenced above is incorporated herein by reference.

The present application relates to optical measurement systems and methods. Periprosthetic fractures may occur for many reasons, including when implants are seated with too great a force or are subjected to high loading in vivo. Alternatively, such fractures can also arise in cases of compromised bone quality. In general, implants fit into bone with some degree of mechanical interference, which is particularly true in Total Hip Arthoplasty (THA) where, following the study in Pagani N R, Varady N H, Chen A F, Rajaee S S, Kavolus J J. Nationwide Analysis of Lower Extremity Periprosthetic Fractures. J Arthroplasty. 2021 January; 36(1):317-324, doi: 10.1016/j.arth.2020.07.050. Epub 2020 Jul. 25. PMID: 32826143, roughly 2% of implant recipients experience a fracture at some point post-operatively.

Seating implants with an appropriate degree of interference and avoiding high bone stress or fracture can be a challenge.

Specific examples of optical measurement systems will now be described with reference to the accompanying drawings, in which:

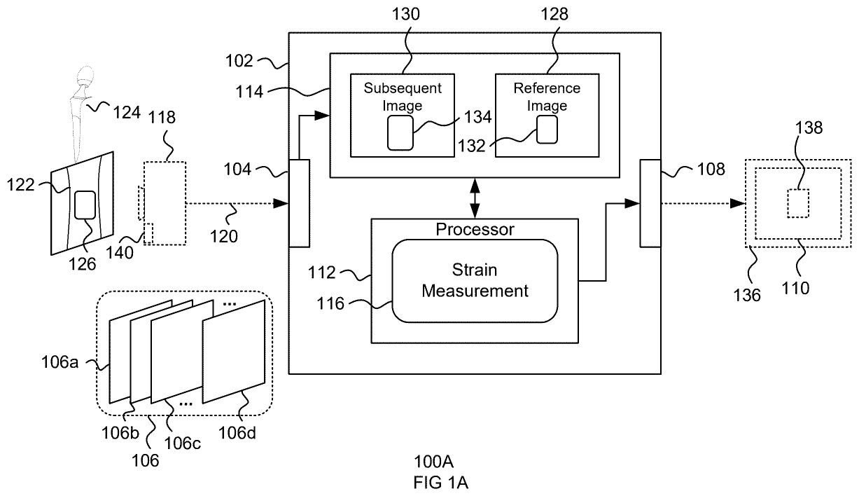

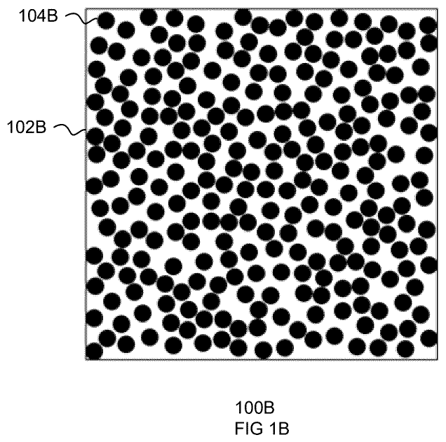

FIG. 1A depicts a view of an example of an optical measurement system;

FIG. 1B illustrates a view of a speckle pattern for use with the optical measurement system;

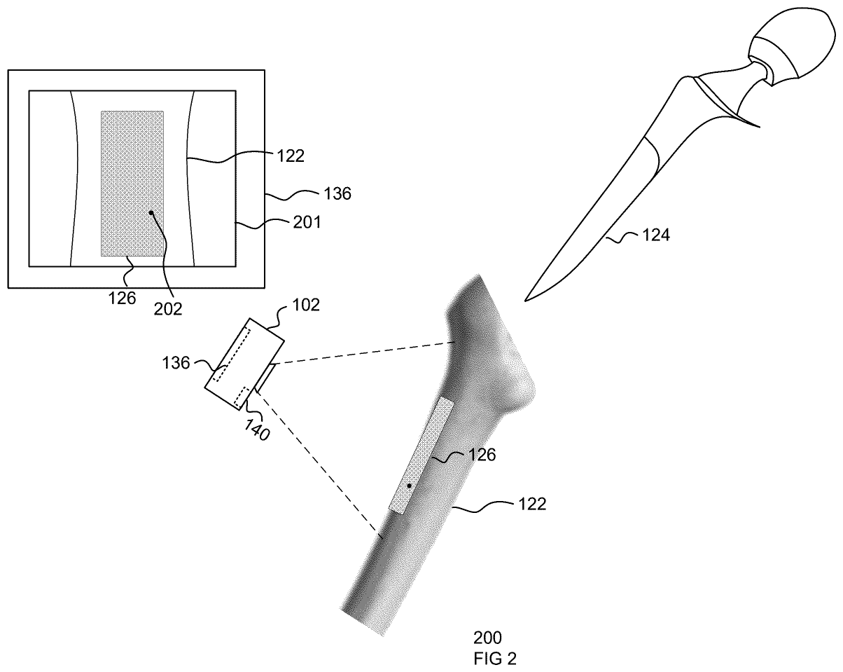

FIG. 2 shows a view of the optical measurement system being used prior to seating an implant in a body part;

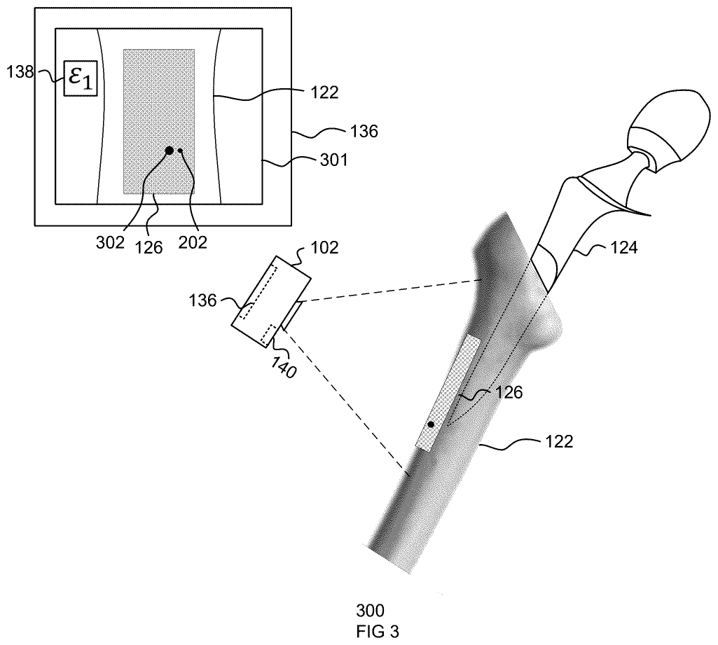

FIG. 3 illustrates a view of the optical measurement system being used to monitor strain during seating the implant into the body part;

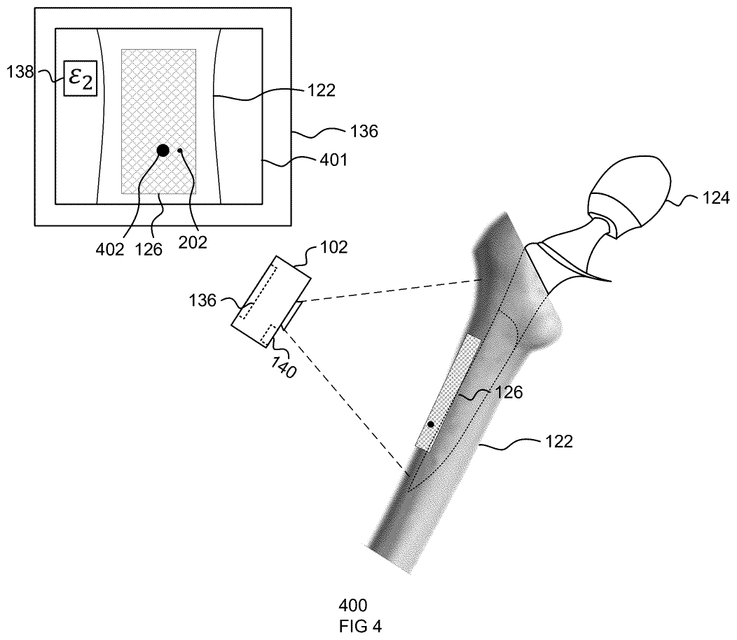

FIG. 4 depicts a view of the optical measurement system being used to monitor strain with the implant being more deeply or possibly fully seated in the body part;

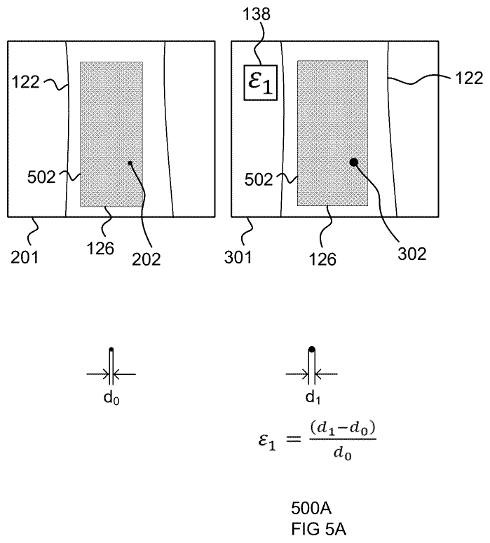

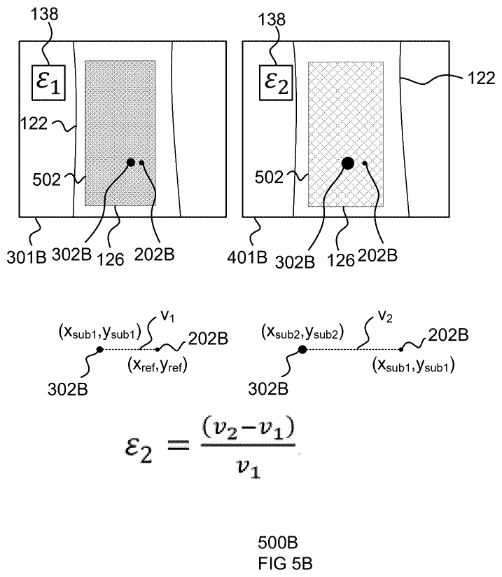

FIG. 5A shows a view of visual data derived from the optical measurement system associated with seating the implant in the body part from which body part strain measurements can be derived;

FIG. 5B shows a view of further visual data derived from the optical measurement system associated with seating the implant in the body part from which body part strain measurements can be derived;

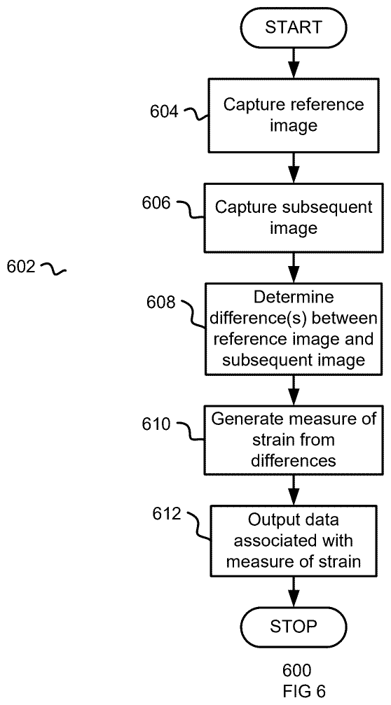

FIG. 6 illustrates a view of a flowchart for processing the visual data to determine a strain measurement;

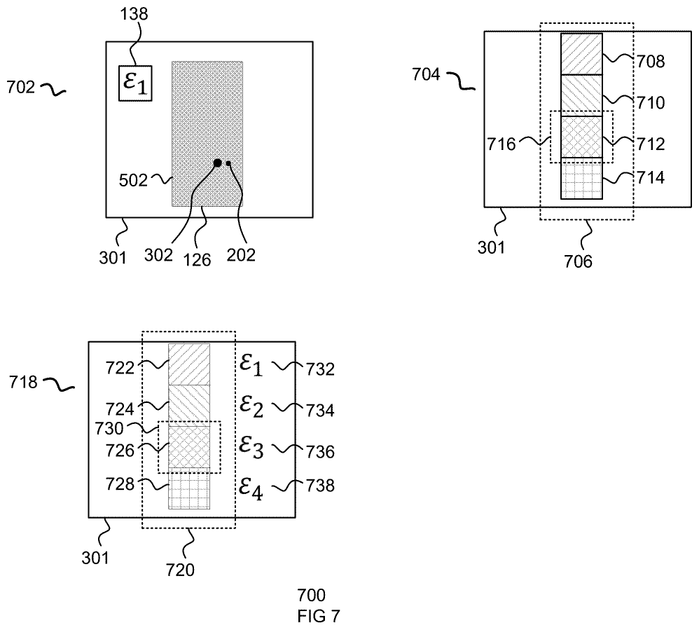

FIG. 7 depicts a view of alternative ways of conveying strain measurement indications relating to the body part;

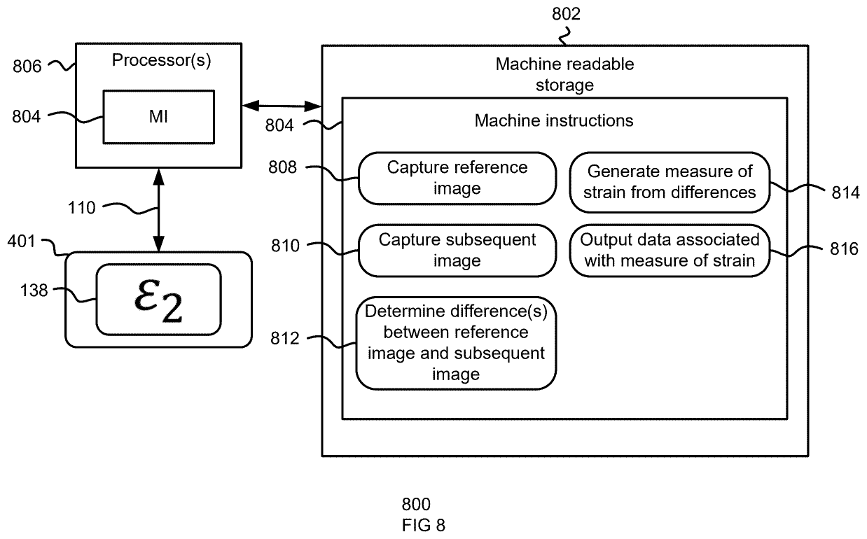

FIG. 8 illustrates a view of a machine-readable medium storing machine instructions for realising examples of the optical measurement system; and

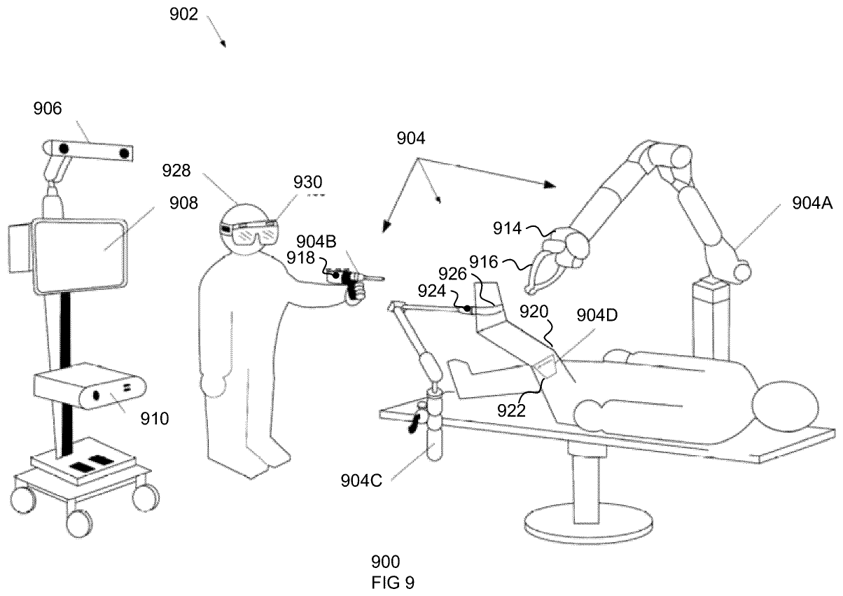

FIG. 9 illustrates an example of Computer Assisted Surgical System (CASS) in which the examples of the optical measurement system can be used.

Referring to FIG. 1A, there is shown a view 100 of an optical measurement system 102. The optical measurement system 102 comprises an input interface 104 for receiving input data 106, and output interface 108 for outputting output data 110, a processor 112 for processing the input data and for generating the output data, and a memory 114 for storing at least one, or both, of the input data and the output data.

The processor 112 can be configurable with software 116 for processing the input data 106 and for generating the output data 110.

In the example depicted in FIG. 1A, the input data 106 comprises a plurality of images temporally consecutive images; four images 106a, 106b, 106c and 106d are shown. The images 106a-d are derived from a camera 118. The camera 118 is shown in dashed-line form, since examples can be realized in which the camera 118 is integral to the optical measurement system 102 or is an external camera that provides output signals 120 to the optical measurement system 102. The output signal 120 comprises the input images 106.

The images 106a-d are of a subject. In the example depicted, the subject is a body part 122. The body part is, in the example shown, a bone such as, for example, a femoral bone or femur. The body part is an example of a target tissue. More specially, the body part can be of the proximal end of the shaft of the femur into which an implant 124 can be seated. In the example depicted, the implant 124 is a femoral prosthesis comprising a femoral head and a femoral stem.

The body part 122 is arranged to bear a reference or indicium 126. Examples can be realized in which the reference or indicium 126 comprises, for example, a speckle pattern, which is described with reference to FIG. 1B. Examples can be realized in which the reference or indicium 126 comprises a set of indicia such as, for example, at least two indicia. The indicia can be any shape, but examples can be realized in which the indicia are dots. Still further examples can be realized in which the reference or indicium 126 is an indentation, scoring or other physical mark on the body part 122. The reference or indicium 126 is such that physical movement of the body part 122 gives rise to a corresponding change or movement in the reference or indicium 126. For example, if the body part expands, the reference or indicium 126 also expands. The characteristics of the speckle pattern can varied. For example, at least one, or both, of: the size of the indicia and the overall coverage of the indicia can be varied. For instance, examples can be realized in which the coverage is 10%, 30%, 50% or some other value. Additionally, or alternatively, to the percentage of coverage options, the dimensions of the indicia can be varied. For instance, the dots may have a diameter of 1 mm, 3 mm, 5 mm or some other dimension. In the specific speckle pattern 102B depicted in FIG. 1B, the dots have a diameter of 4.5 mm.

The processor 112 and software 116 are arranged to receive and to store in the memory 114 a reference image 128 and a temporally subsequent image 130 selected from the images 106a-d. The reference image 128 comprises a representation 132 of the reference or indicium 126. The subsequent image 128 comprises a representation 134 of the reference or indicium 126. The reference image 128 is taken at a time t₁. The subsequent image 130 is taken at a later time t₂. The subsequent image 130 is taken during seating the implant 124. It can be seen that the representations 132 and 134 of the reference or indicium 126 are different. In the example depicted, the representation 134 in the subsequent image 130 has an expanded state compared to the representation 132 in the reference image 128.

The processor 112 and software 116 are arranged to compare the reference image 128 and the subsequent image 130 to identify any differences in the reference or indicium 126. The identified differences are used to determine a physical characteristic associated with the body part 122. In the example depicted, the physical characteristic is the strain to which the body part 122 is subjected as a consequence of inserting the implant 124 or the implant 124 having been inserted. For example, the femoral stem of a femoral prosthesis can rely on a frictional fit to remain seated within the femur. The process of seating the femoral prosthesis, and the femoral prosthesis being seated, expands the proximal end of the shaft of the femur.

Once a strain measurement has been determined, the output data 110 representing that strain measurement is output via the output interface 108. The output data 110 can be output for further processing. Alternatively, or additionally, the output data 110 can be output for display on a display 136. The displayed output data 110 comprises data 138 representing the determined strain measurement. A surgeon, or other member of operating room staff, can use the display to note the current strain to which the body part 122 is subject.

The output data 110 comprising the data 138 representing that strain measurement can comprise at least one, or more than one, of the following taken jointly and severally in any and all permutations: a numerical indication representing the measure of strain, a graphical representation of the strain measurement, a colour indicative of the strain, a contour plot (with or without labels indicative of strain) showing the distribution or variation of the strain across the bodily part, or at least across a region of interest of the bodily part. Examples can be realized in which the region of interest is defined by the region covered by the reference or indicium 126.

The display 136 and the displayed output data 110 containing the data 138 indicative of the strain measurement are all shown in dashed-line form to indicate that the foregoing may or may not form an integral part of the optical measurement system 102. Examples can be realized in which the display 136 forms an integral part of the optical measurement system 102. Alternatively, examples can be realized in which the output data is merely output for display or further processing by an entity that does not form an integral part of the optical measurement system 102.

Examples can be realized in which both the display 136 and the camera 118 form integral parts of the optical measurement system 102. In such an example, an image 106a-106d presently within the field of view (not shown) of the camera 118 can be displayed on the display 136. Examples can be realized in which the output data 110 can be displayed independently of any image of the images 106a-d, or in conjunction with any such image of the images 106a-d. Examples can be realized in which the output data 110 is displayed in an overlaid or overlapping relationship with an image of the subject 122 and reference or indicium 126. Such an integrated optical measurement system comprising at least one, or both, of: the camera 118 and display 136, can be realized as, or as part of, a handheld device.

The camera 118 can further comprise a range finder 140. The range finder 140 determines the distance of the camera to the body part 122. The purpose of knowing the distance is so that changes in the image, in particular, in the reference or indicium 126, can be compensated for due to variations in proximity of the camera 118 to the subject. Alternatively, or additionally, the range finder 140 can be used to ensure that the images 106a-d are acquired at the same range from the subject. Still further, rather than using a range finder 140, a reference object can be placed within the field of view of the camera 118. The reference object has known physical characteristics. For example, the reference object can have known dimensions. The known dimensions can be used as a reference when determining the size of other objects within a captured image such as, for example, the dots described below.

FIG. 1B illustrates a view 100B of an example of a reference or indicium 126 for use with the optical measurement system 102. In the example depicted, the reference or indicium 126 comprises a speckle pattern 102B. The speckle pattern 102B comprises a set of randomly distributed indicia 104B of a predetermined size. In the example shown, the indicia comprise randomly distributed dots.

FIG. 2 shows a view 200 of the optical measurement system 102 being used prior to seating the implant in a body part 122. The optical measurement system 102 is shown as comprising both the display 136 and the camera 118 as integral entities. The optical measurement system 102 depicts an image 201 comprising the body part 122 and the reference or indicium 126. The image 201 can be an example of a reference image or of a subsequent image. Also shown is the femoral prosthesis 124 prior to the surgical seating procedure commencing. As described above with reference to FIG. 1B, the reference or indicium 126 can be, for example, a pattern such as a random speckle pattern, a regular or repeating pattern, or can comprise a set of marks. Such a set of marks can comprise a single mark or a number of marks. In the illustrated example, the set of marks comprises a single dot 202.

The implant 124 is poised for seating. Therefore, the femur 122 will not be subject to any strain associated with the implant 124. Consequently, the image of the reference or indicium will not be changed or otherwise distorted due to the presence of strain within the femur 122. An image of such an unstrained body part can be an example of the above described reference image 128. However, a reference image does not have to be an image of an unstrained body part. Examples can be realized in which the reference image is captured merely temporally before the subsequent image 130.

FIG. 3 illustrates a view 300 of the optical measurement system 102 used to monitor strain during seating the implant 124 into the body part 122. The camera 118 and the display 136 are integral to the optical measurement system 102. Again, the display 136 shows an image 301 of the body part 122 and the reference or indicium 126. The image 301 can be an example of a reference image relative to a subsequent image or can be a subsequent image relative to a previous image. The femoral prosthesis 124 is shown as being partially seated. Again, the reference or indicium 126 can be, for example, a pattern such as a regular or repeating pattern, or can comprise a set of marks. Such a set of marks can comprise a single mark or a number of marks. In the illustrated example, the set of marks comprises a single dot 302, which corresponds to the above described dot 202. It can be appreciated that the dot 302, while corresponding to the above described dot 202, has at least one, or more, different physical characteristics compared to the dot 202. In the example depicted, the dot 302 comprises at least one, or both, of: a larger radius/diameter and/or has undergone a translation. In the example shown, the dot 302 is in an expanded state relative to the previous dot 202.

It can be appreciated that the reference or indicium 126 has an increased width relative the reference or indicium depicted in FIG. 2. The increased width has been exaggerated for the purpose of illustration. In FIG. 2, the width is 15 mm, whereas in FIG. 3 the width is 17 mm. The increase in width follows as a consequence of inserting or seating the implant 124. In the particular example depicted in FIG. 3, the femoral stem of the femoral prosthesis 124 will exert a radially outwardly directed force on the femur shaft.

The implant 124 is in the process of being seated and is shown as being partially seated. Therefore, the femur 122 will be subject to strain associated with the implant 124. Consequently, the image of the reference or indicium will be changed or otherwise distorted due to the presence of strain within the femur 122. An image of such a strained body part can be an example of the above described subsequent image 130. It can be appreciated that the subsequent image has, firstly, been acquired at a later time relative to the reference image 128 and, secondly, has been acquired post a surgical event. In the example depicted, the surgical event will have been actuating the femoral prosthesis 124 to drive it into the current position.

As indicated above, the difference between the reference image 128 and the subsequent image 130 can be used to determine the strain to which the bone is subject due to the implant 124.

In the example depicted, the display 136 can display the indication 138 of the strain within body part 122. In the example shown in FIG. 3, the indication 138 comprises a numerical value, ε₁, indicating the strain measured.

FIG. 4 depicts a view 400 of the optical measurement system used to monitor strain within the femur when the implant is more deeply or possibly fully seated. The display 136 shows an image 401 of the body part 122 and the reference or indicium 126. The image 401 is an example of a subsequent image, but could equally well be a reference image relative to a temporally subsequent image. The femoral prosthesis 124 is shown as being more deeply or possibly fully seated. As before, the reference or indicium 126 can be, for example, a pattern such as a regular or repeating pattern, or can comprise a set of marks. Such a set of marks can comprise a single mark or a number of marks. In the illustrated example, the set of marks comprises a single dot 402, which corresponds to the above described dot 302 at a later point in time. It can be appreciated that the dot 402, while corresponding to the above described dot 302, has at least one, or more, different physical characteristics compared to the dot 302. In the example depicted, the dot 402 comprises at least one, or both, of: a larger radius/diameter and/or has undergone a translation.

It can be appreciated that the reference or indicium 126 has an increased width relative the reference or indicium depicted in FIG. 3. The increased width has been exaggerated for the purpose of illustration. In FIG. 3, the width is 17 mm, whereas in FIG. 4 the width is 18 mm. The increase in width follows as a consequence of further inserting and finally seating the implant 124. In the particular example depicted in FIG. 4, the femoral stem of the femoral prosthesis 124 will continue to exert a radially outwardly directed force on femur shaft.

The implant 124 is shown as being more deeply or possibly fully being seated. Therefore, the femur 122 will be subject to strain associated with the implant 124. Consequently, the image 401 of the reference or indicium will be changed or otherwise distorted due to the presence of strain within the femur 122. An image of such a strained body part can be an example of the above described subsequent image 130. It can be appreciated that the subsequent image 401 has, firstly, been acquired at a later time relative to the reference image 128 and, secondly, has been acquired post a surgical event. In the example depicted, the surgical event will have been actuating the femoral prosthesis 124 to finally seat the implant 124.

As indicated above, the difference between the reference image and the subsequent image can be used to determine the strain to which the bone is subject due to the implant 124.

In the example depicted, the display 136 can display the indication 138 of the strain within body part 122. In the example shown in FIG. 4, the indication 138 comprises a numerical value, ε₂, indicating the strain measured. In the example shown, it is anticipated that ε₂>ε₁.

FIG. 5A shows a view 500A of visual data derived from the optical measurement system 102 associated with seating the implant in the body part from which body part strain measurements have been derived.

It will be appreciated that the references or indicia are merely schematic for the purposes of explanation and illustration. As indicated above with reference to FIG. 1B, examples can be realized in which speckle patterns are used for strain measurement.

Assume that the reference or indicium 126 is a single dot such as dot 202 and that the first image 201 is the reference image. The optical measurement system 102 is arranged to determine a respective diameter, do, of the dot 202.

Post actuating the implant 124 during the process of seating the implant 124, or having fully seated the implant 124, a temporally subsequent image such as, for example, image 301, is captured by the camera 118. Inserting or otherwise seating the implant 124 will cause the body part 122, which is the femur in the present example, to expand. That expansion will lead to a change in the reference or indicium 126. In the present case, the change in the indicium is shown as being a change in diameter. The optical measurement system 102 is arranged to determine a respective diameter, d₁, of the dot 302, which is an expansion of the previous dot 202.

In general, strain is given by the ratio: a change in dimension/original dimension. Therefore, in the present case, the strain, ε₁, experienced by the femur during the process of seating the implant 124 is given by:

$${ε}_{1} = \frac{( {d}_{1} - {d}_{0} )}{{d}_{0}} .$$

Post further actuating the implant 124 until the implant 124 is more deeply or possibly fully seated, a further temporally subsequent image (not shown) can be captured by the camera 118. Again, having the implant 124 more deeply or possibly fully seated will cause the body part 122 to expand further. That further expansion will lead to a further change in the reference or indicium 126. In the present case, assume that the further change in the indicium is a continued expansion in diameter. The optical measurement system 102 would be arranged to determine a respective diameter, d₂, of the newly expanded dot (not shown), which would be an expansion of the previous dot 302.

Therefore, at such a point in time, the strain, ε₂, experienced by the femur when the implant 124 is more deeply or possibly fully seated would be given by:

$${ε}_{2} = \frac{( {d}_{2} - {d}_{1} )}{{d}_{1}} .$$

Although the above examples of determining strain measurements rely on detecting a change in a physical characteristic of a reference, example are not limited to such arrangements. Examples can be realized in which a change in some other dimension can be used to determine the strain.

Although the above examples have been described with reference to using a subsequent image and a temporally immediately preceding image as the reference image, examples are not limited to such an arrangement. Examples can be realized in which any pair of temporally successive images are used as a reference image and a subsequent image. For example, the initial image 201 could be used as a reference image and the final image (not shown) could be used as the temporally subsequent image. In such an example, the strain, ε₂, experienced by the femur when the implant 124 is more deeply or possibly fully seated would be given by:

$${ε}_{2} = \frac{( {d}_{2} - {d}_{0} )}{{d}_{0}} .$$

Accordingly, examples can be realized in which a change in, for example, position or distance of, or associated with, the reference or indicium can be used as the basis for determining the strain.

Therefore, referring to FIG. 5B, there is shown a view 500B of a pair of reference dots 202B and 302B. It can be seen that a first dot 202B of the pair of reference dots in the first reference image 301B is separated by a distance, or vector, v₁, from the second dot 302B of the pair of reference dots given, in a two-dimensional case, by the difference between the position of the first dot 202B, which is (x_{ref},y_{ref}), and the position of the second dot 302B, which is (x_{sub1},y_{sub1}).

FIG. 5B depicts a further subsequent image 401B. Referring to the second subsequent image 401B, the separation between the first dot 202B and the second dot 302B change as reflected by a further vector, v₂, derived from the position of the first dot 202B, which is (x_{sub1},y_{sub1}), and the position of the second dot 302B, which is (x_{sub2},y_{sub2}). It should be noted that the position of the first dot 202B in the second image 401B has been generalised to (x_{sub1},y_{sub1}). However, examples can be realized in which (x_{sub1},y_{sub1})=(x_{ref},y_{ref}).

Therefore, the strain, ε₂, is given by:

$${ε}_{2} = \frac{( {v}_{2} - {v}_{1} )}{{v}_{1}} .$$

Although the examples described with reference to FIGS. 5A and 5B use a change in diameter, or other physical characteristic of an indicium, and a change in separation between indicia as the basis for respective strain measurements, examples are not limited thereto. Examples can be realized in which a combination of both a change in a physical characteristic of an indicium and a change in relative position of indicia are both used to calculate a measurement of strain. For example, a simple average of the measurements determined from FIGS. 5A and 5B could be used as the basis of measuring strain.

Examples can be realized in which the camera 118 is a stereoscopic camera, or in which multiple cameras are used to capture the reference image and subsequent image. In such cases, 3D vectors can be used to determine strain, which will take into account any out of plane movement of the body part 122. Using such a stereoscopic camera or using multiple cameras supports realising examples without the above described range finder 140.

Alternatively, as described above with reference to FIG. 1B, a measurement of strain can be derived using a speckle pattern as the reference or indicium, which are marked generically in FIG. 5 as 502. The optical measurement system 102 can comprise a laser interferometry system in which a laser is used to illuminate a region of interest of the body part 122, and in which the camera 118 is used to record the laser speckle pattern. Such a laser is divided into two beams by a beam splitter. A first beam, which is also known as an object beam, is directed to the object to be measured, while the second beam, which is also known as a reference beam, is used as a reference. The optical measurement system 102 recombines the first and second beams to produce an interference pattern. The interference pattern varies according to the phase differences between the first and second beams. When the body part 122 is deformed, the phase difference changes, which, in turns, changes the interference pattern. By processing temporally consecutive interference patterns, that is, a reference interference pattern and a subsequent interference pattern, at two different points in time, a phase displacement gradient map can be realized from which strain can be calculated. Such a reference interference pattern is an example of the above-described reference image 128. Such a subsequent interference pattern is an example of the above-described temporally subsequent image 130.

FIG. 6 illustrates a view 600 of a flowchart 602 for processing image data to determine a strain measurement. At 604, a reference image 128 is captured at a respective time, t₁. Following a surgical action, such as, for example, actuating the implant during the process of seating the implant 124, a subsequent image 130 is captured at 606 at a respective time, t₂. The differences between the reference image and the subsequent image are determined at 608. At 610, a measure of strain is derived from the differences established at 608. Output data associated, with or representative of, the measure of strain are output at 612 for either display or further processing.

In the case of non-contact speckle pattern interferometry, it will be appreciated that the speckle pattern per se forms the reference or indicium 126. However, in other examples, the reference or indicium 126 can be applied to the body part 122 in advance of capturing any images or the body part 122 per se may bear features forming the reference or indicium 126. For example, the surface of the body part 122 may comprise a natural variation that can be used as a reference or indicium 122. For instance, the natural variability of a bone surface could serve as the reference or indicium 126. Alternatively, the surface of a body part could be intentionally patterned to bear such a reference or indicium 126. For instance, divots, scored marks, burn marks from a tool such as a bovie, and the like could be applied to the body part 122 to serve as a reference or indicium 126. Still further, a set of devices could be applied to the surface of the body part 122 to form the reference or indicium 126. The set of devices can comprise one or more than one device. A device of such a set can comprise an attachment to the body part such as, for example, a pin attached to the femur or other body part of interest. Still further, examples can be realized in which the reference or indicium 126 is applied to the body part 122 in the form of a mesh or paper bearing indicia. Such a paper or mesh may comprise, for instance, a speckle pattern or other random pattern. The paper or mesh may be affixed to the body part 122 using an adhesive such as, for example, a cyanoacrylate adhesive.

The reference or indicium 126 may be used in Digital Image Correlation to determine the strain within the body part 122.

FIG. 7 depicts a view 700 of possible outputs 702 to 718 providing indications of strain within the body part 122 with the implant 124 in-situ. A first output has been described above with reference to FIG. 5. The indication of strain 138 is given as a single dimensionless number.

A second output 704 presents on the display 301 a set of indicia 706 in which each indicium 708 to 714 forms a scale of different strain measurements extending from one indication of strain 708 to another indication of strain 714. The indications of strain 708 and 714 can represents a maximum strain 708 to a minimum strain 714 associated with the maximum and minimum strains measurable by the optical measurement system 102 or the maximum and minimum strains measured thus far by the optical measurement system 102. A current strain value 712 representing the latest strain measurement can be highlighted via a highlighting indicium 716 on the scale. Highlighting the current strain value 712 using the highlighting indicium 716 can take many different forms such as, for example, varying the intensity, colour, size, fill pattern, or some other graphical characteristic of the scale.

A third output 718 presents on the display 301 a set of indicia 720 in which each indicium 722 to 728 forms a scale of different strain measurements extending from one indication of strain 722 to another indication of strain 728. The indications of strain 722 and 728 can represents a maximum strain 722 to a minimum strain 728 associated with the maximum and minimum strains measurable by the optical measurement system 102 or the maximum and minimum strains measured thus far by the optical measurement system 102. A current strain value 726 representing the latest strain measurement can be highlighted via a highlighting indicium 730 on the scale. Highlighting the current strain value 726 using the highlighting indicium 730 can take many different forms such as, for example, varying the intensity, colour, size, fill pattern, or some other graphical characteristic of the scale. Examples can be realized in which one or more of the indicia 722 to 728 can have an adjacently displayed respective numerical strain measurement value 732 to 728. Alternatively, examples can be realized in which only the currently strain value 726 has a respective numeral strain measurement value 736 adjacently depicted.

FIG. 8 illustrates a view 800 of a machine-readable storage 802 storing machine instructions 804 for realising examples of the optical measurement system 102.

The machine instructions 804 are instructions arranged, when processed by a processor 806, to implement the method of FIG. 6.

Accordingly, the machine instructions 804 comprise:

Instructions 808 to capture a reference image 128 at a respective time, t₁;

Instructions 810 to capture a temporally subsequent image 130 at a respective time, t₂, following a surgical action, such as, for example, actuating the implant during the process of seating the implant 124;

Instructions 812 to determine the differences between the reference image 128 and the subsequent image 130;

Instructions 814 to derived a measure of strain from the established differences; and

Instructions 816 to output data 110 associated, with or representative of, the measure of strain for either display or further processing.

The reference image 128 and the temporally subsequent image 130 can be taken at respective points in time in response to an event, such as, for example, actuating or otherwise triggering the camera 118 to capture an image. Alternatively, the camera 118 can be arranged to generate a video stream comprising many temporally successive images. The reference image 128 and a temporally subsequent image 130 can be derived from the video stream to give a current measurement of strain. Alternatively, or additionally, a current indication of strain can be continuously displayed using respective pairs of reference and subsequent images of the video stream.

Referring to FIG. 9, there is shown a view 900 of a CASS 902 according to an example. In the example depicted, the CASS 902 is arranged to aid surgeons in performing orthopedic surgical procedures such as, for example, an arthroplasty (e.g., total knee arthroplasty (TKA)) or total hip arthroplasty (THA). An effector platform 904 positions surgical tools relative to a patient during surgery. The effector platform 904 can comprise a robotic arm 904A. The effector platform 904, in the case of, for example, knee surgery, may include an end effector 904B that holds surgical tools or instruments during their use. Examples can be realized in which the end effector 904B can be realized as an optical measurement system 102 as described and/or as claimed herein. The effector platform 904 can include a limb positioner 904C for positioning the patient's limbs during surgery. Resection equipment (not shown in FIG. 9) performs bone or tissue resection using, for example, mechanical, ultrasonic, or laser techniques. The effector platform 904 can also include a cutting guide or jig 904D that is used to guide saws or drills used to resect tissue during surgery. Such cutting guides 904D can be formed integrally as part of the effector platform 904 or as part of the robotic arm 904A, or cutting guides can be separate structures that can be matingly and/or removably attached to the effector platform 904 or the robotic arm 904A.

The CASS 902 comprises a tracking system 906 that uses one or more sensors to collect real-time position data that locates the patient's anatomy and surgical instruments. Any suitable tracking system can be used for tracking surgical objects and patient anatomy in the surgical theatre. For example, a combination of infrared (IR) and visible light cameras can be used in an array. Such a tracking system 906 can use the EMR retro-reflected from any of the retro-reflectors described and/or claimed herein to determine real-time position data that locates at least one, or both, of the patient's anatomy and surgical instruments.

Accordingly, the CASS 902 shown in FIG. 9 depicts a number of retro-reflectors. The retro-reflectors can be any of the retro-reflectors described and/or claimed herein. The retro-reflectors can be placed on objects or body parts to be tracked or for which respective positions are to be determined. For example, a first retro-reflector 914 is situated on the robot arm 904A. Knowing the position of the retro-reflector 914 can allow, for example, the position of the actuator 916 of the robot arm 904A to be determined. A second retro-reflector 918 is placed on the handheld tool 904B to allow the position of the to be determined and/or tracked in 3D space. A third retro-reflector 920 can be situated relative to the jig 904D. At least a fourth retro-reflector 922 can be used to determine not only the position of the jig 904D, but also the attitude in 3D space of the jig 904D. A sixth retro-reflector 924 can be placed on the Limb Positioner 904C to assist in determining the position of a respective distal actuator 926 for holding a limb.

The registration process that registers the CASS 902 to the relevant anatomy of the patient can also involve the use of anatomical landmarks, such as landmarks on a bone or cartilage. For example, the CASS 902 can include a 3D model of the relevant bone or joint and the surgeon can intraoperatively collect data regarding the location of bony landmarks on the patient's actual bone using a probe that is connected to the CASS. Alternatively, the CASS 902 can construct a 3D model of the bone or joint without pre-operative image data by using location data of bony landmarks and the bone surface that are collected by the surgeon using a CASS probe or other means.

A tissue navigation system (not shown in FIG. 9) provides the surgeon with intraoperative, real-time visualization for the patient's bone, cartilage, muscle, nervous, and/or vascular tissues surrounding the surgical area.

The CASS 902 comprises a display 908 to provide graphical user interfaces (GUIs) that display images collected by the Tissue Navigation System as well other information relevant to the surgery to a surgeon or other operating theatre staff 928. The display 908 can be an example of the above described display 136. For example, the display 908 can overlay image information collected from various modalities (e.g., CT, MRI, 9-ray, fluorescent, ultrasound, etc.) collected pre-operatively or intra-operatively to give the surgeon various views of the patient's anatomy as well as real-time conditions. A surgical computer 910 provides control instructions to various components of the CASS 902, collects data from those components, and provides general processing for various data needed during surgery. In the example depicted in FIG. 9, the surgeon 928 is shown as wearing protective eye-ear 930.

In some examples, the camera 118 may be integral with the tracking system 906. Example embodiments may incorporate the display 130 in whole or in part into the display 908. The optical measurement system 102 may be integral with the surgical computer 910. In such examples, the CASS 902 can be equipped to accomplish the methods and include the systems disclosed in FIGS. 1-8 and accompanying text.

Examples can be realized according to the following clauses:

Clause 1: An intraoperative optical strain measurement system to determine strain within a target tissue to receive an implant; the system comprising: an input for receiving temporally consecutive images of the target issue; the temporally consecutive images comprising: at least one reference image and at least one subsequent image taken after the at least one reference image; a strain measurement processor comprising: measurement calculation circuitry, responsive to a data associated with a difference between the at least one reference image and the at least one subsequent image, to determine strain measurement data indicative of strain within the target tissue, and an output for outputting strain measurement data associated the measure of strain.

Clause 2: The system of clause 1, comprising a camera to generate the consecutive images of the target issue.

Clause 3: The system of clause 2, in which the camera is a stereoscopic camera.

Clause 4: The system of any preceding clause, in which the strain measurement processor is arranged to generate the strain measurement data by identifying differences between the at least one reference image and the at least one subsequent image.

Clause 5: The system any preceding clause, in which the strain measurement processor is arranged to generate at least one 2D vector (xₛ,yₛ)=(x_{sub}−x_{ref},y_{sub}−y_{ref}) or at least one 3D vector (xₛ,yₛ,zₛ)=(x_{sub}−x_{ref},y_{sub}−y_{ref},z_{sub}−z_{ref}) associated with movement of at least one indicium common to the at least one reference image and the at least one subsequent image.

Clause 6: The system of clause 5, in which the at least one indicium common to the at least one reference image and the at least one subsequent image comprises: at least two indicia common to the at least one reference image and the at least one subsequent image, optionally, in which the at least one indicium common to the at least one reference image and the at least one subsequent image comprises a speckle pattern.

Clause 7: The system of either of clauses 5 and 6, in which the strain measurement processor derives the strain measurement data of the target tissue from the at one 2D or 3D vector.

Clause 8: The system of any preceding clause, comprising at least one output device for displaying said strain measurement data.

Clause 9: The system of clause 8, in which the strain measurement data represents a 2D or 3D map of variation of strain across a respective region.

Clause 10: The system of clause 9, in which the 2D or 3D map is arranged to be displayed in an overlay registered relationship with an image of the target tissue material.

Clause 11: Machine-readable instructions arranged, when processed, to realize an intraoperative optical strain measurement system to determine strain within a target tissue; the instructions comprising: instructions for receiving temporally consecutive images of the target tissue; the temporally consecutive images comprising: at least one reference image bearing at least one reference indicium and at least one subsequent image, taken after the at least one reference image, bearing the at least one reference indicium; instructions to: generate strain measurement data, based on data associated with a difference the at least one reference image and the at least one subsequent image, indicative of strain of the target tissue, and instructions for outputting the strain measurement data.

Clause 12: Machine-readable instructions of clause 11, comprising instructions to control a camera to generate the consecutive images of the target tissue material.

Clause 13: Machine-readable instructions of either of clauses 11 to 12, comprising instructions to control at least one display device for displaying the strain measurement data.

Clause 14: Machine-readable instructions of clause 13, in which the strain measurement data represents at least one or more than one of the following taken jointly and severally in any and all permutations: an indicium indicative of the strain measurement data, a strain scale indicative of a range of strains, a numerical indication strain, a 2D or 3D map of variation of strain across a respective region of the target tissue material.

Clause 15: Machine-readable instructions of clause 14, in which the 2D or 3D map is arranged to be displayed in an overlaid registered relationship with an image of the target tissue material.

Clause 16: Machine-readable instructions of any of clauses 11 to 15, in which the instructions to generate strain measurement data is arranged to generate the strain measurement data by identifying differences between the at least one reference image and the at least one subsequent image.

Clause 17: Machine-readable instructions any of clauses 11 to 16, in which the instructions to generate the strain measurement data is arranged to generate at least one 2D vector (xₛ,yₛ)=(x_{sub}−x_{ref},y_{sub}−y_{ref}) or at least one 3D vector (xₛ,yₛ,zₛ)=(x_{sub}−x_{ref},y_{sub}−y_{ref},z_{sub}−z_{ref}) associated with movement of the at least one reference indicium common to the at least one reference image and the at least one subsequent image.

Clause 18: Machine-readable instructions of any of clauses 11 to 17, in which the at least one indicium common to the at least one reference image and the at least one subsequent image comprises: at least two indicia common to the at least one reference image and the at least one subsequent image; optionally, in which the at least one indicium common to the at least one reference image and the at least one subsequent image comprises a speckle pattern.

Clause 19: Machine-readable instructions of either of clauses 17 and 18, in which the instructions to generate the strain measurement data derives a measure of strain of the target material from the at one 2D or 3D vector.

Clause 20: Machine-readable storage storing machine-readable instructions of any of clauses 11 to 19.

Clause 21: An intraoperative method of determining a physical characteristic within a body member; the method comprising: creating at least two reference indicia on the body member in a predetermined region of interest of the body member; the at least two reference indicia being spaced-apart by a respective distance; capturing a reference image comprising the at least two reference indicia; performing a surgical action as part of a surgical procedure that subjects the body member to strain in the region of interest of the body member; capturing a further image comprising the at least two reference indicia; and deriving a measure of strain within the body member from the reference image and the further image.

Clause 22: The method of clause 21, in which deriving a measure of strain within the body member from the reference image and the further image comprises comparing the change in separation of the at least two reference indica between the reference image and the further image, and calculating a measure of strain from the change in separation of the at least two reference indicia and the respective distance.

Clause 23: An intraoperative optical measurement system to measure a strain to avoid a periprosthetic fracture within a bone to receive an implant; the system comprising: an input for receiving temporally consecutive images of the bone; the temporally consecutive images comprising: at least one reference image bearing at least one reference indicium; and at least one subsequent image, taken after the at least one reference image and post a surgical action associated with the implant, bearing the at least one reference indicium; a strain measurement processor arranged to: determine strain measurement data associated with strain of the bone from data associated with a difference between the at least one reference image and the at least one subsequent image; and an output for outputting strain measurement data.

Clause 24: An intraoperative method of determining a measure of strain within a body member; the method comprising: illuminating the body member in a region of interest of the body member with an object beam speckle pattern derived from a laser; capturing a reference image comprising a combination of the object bean speckle pattern reflected from the region of interest of the body member with a reference beam speckle pattern derived from the laser; performing a surgical action as part of a surgical procedure that subjects the body member to strain in the region of interest of the body member; capturing a temporally subsequent image comprising a further combination of the object bean speckle pattern reflected from the region of interest of the body member with a respective reference beam speckle pattern derived from the laser; and deriving a measure of strain within the body member from the reference image and the temporally subsequent image.

Clause 25: The intraoperative method of clause 24, in which deriving a measure of strain within the body member from the reference image and the temporally subsequent image comprises: generating a phase displacement map, and determining a measure of strain from the phase displacement map.

1. An intraoperative optical strain measurement system to determine strain within a target tissue to receive an implant; the system comprising:
a. an input for receiving temporally consecutive images of the target issue; the temporally consecutive images comprising:
i. at least one reference image and
ii. at least one subsequent image taken after the at least one reference image;
b. a strain measurement processor comprising:
i. measurement calculation circuitry, responsive to a data associated with a difference between the at least one reference image and the at least one subsequent image, to determine strain measurement data indicative of strain within the target tissue, and
c. an output device for outputting strain measurement data associated the measure of strain.

2. The system of claim 1, comprising a camera to generate the consecutive images of the target issue.

3. The system of claim 2, in which the camera is a stereoscopic camera.

4. The system of claim 1, in which the strain measurement processor is arranged to generate the strain measurement data by identifying differences between the at least one reference image and the at least one subsequent image.

5. The system of claim 1, in which the strain measurement processor is arranged to generate at least one 2D vector (xₛ,yₛ)=(x_{sub}−x_{ref},y_{sub}−y_{ref}) or at least one 3D vector (xₛ,yₛ,zₛ)=(x_{sub}−x_{ref},y_{sub}−y_{ref},z_{sub}−z_{ref}) associated with movement of at least one indicium common to the at least one reference image and the at least one subsequent image.

6. The system of claim 5, in which the at least one indicium common to the at least one reference image and the at least one subsequent image comprises:
a. at least two indicia common to the at least one reference image and the at least one subsequent image, optionally, in which the at least one indicium common to the at least one reference image and the at least one subsequent image comprises a speckle pattern.

7. The system of claim 5, in which the strain measurement processor derives the strain measurement data of the target tissue from the at least one 2D or 3D vector.

8. The system of claim 1, in which the strain measurement data represents a 2D or 3D map of variation of strain across a respective region.

9. The system of claim 8, in which the 2D or 3D map is arranged to be displayed in an overlay registered relationship with an image of the target tissue material.

10. Machine-readable instructions arranged, when processed, to realize an intraoperative optical strain measurement system to determine strain within a target tissue; the instructions comprising:
a. instructions for receiving temporally consecutive images of the target tissue; the temporally consecutive images comprising:
i. at least one reference image bearing at least one reference indicium and
ii. at least one subsequent image, taken after the at least one reference image, bearing the at least one reference indicium;
b. instructions to:
i. generate strain measurement data, based on data associated with a difference the at least one reference image and the at least one subsequent image, indicative of strain of the target tissue, and
c. instructions for outputting the strain measurement data.

11. Machine-readable instructions of claim 10, comprising instructions to control a camera to generate the consecutive images of the target tissue material.

12. Machine-readable instructions of claim 10, comprising instructions to control at least one display device for displaying the strain measurement data.

13. Machine-readable instructions of claim 12, in which the strain measurement data represents at least one or more than one of the following taken jointly and severally in any and all permutations: an indicium indicative of the strain measurement data, a strain scale indicative of a range of strains, a numerical indication strain, a 2D or 3D map of variation of strain across a respective region of the target tissue material.

14. Machine-readable instructions of claim 13, in which the 2D or 3D map is arranged to be displayed in an overlaid registered relationship with an image of the target tissue material.

15. Machine-readable instructions of any of claim 10, in which the instructions to generate strain measurement data is arranged to generate the strain measurement data by identifying differences between the at least one reference image and the at least one subsequent image.

16. Machine-readable instructions any of claim 10, in which the instructions to generate the strain measurement data is arranged to generate at least one 2D vector (xₛ,yₛ)=(x_{sub}−x_{ref},y_{sub}−y_{ref}) or at least one 3D vector (xₛ,yₛ,zₛ)=(x_{sub}−x_{ref},y_{sub}−y_{ref},z_{sub}−z_{ref}) associated with movement of the at least one reference indicium common to the at least one reference image and the at least one subsequent image.

17. Machine-readable instructions of claim 10, in which the at least one indicium common to the at least one reference image and the at least one subsequent image comprises:
a. at least two indicia common to the at least one reference image and the at least one subsequent image; optionally, in which the at least one indicium common to the at least one reference image and the at least one subsequent image comprises a speckle pattern.

18. Machine-readable instructions of claim 16, in which the instructions to generate the strain measurement data derives a measure of strain of the target material from the at one 2D or 3D vector.

19. An intraoperative method of determining a physical characteristic within a body member; the method comprising:
a. creating at least two reference indicia on the body member in a predetermined region of interest of the body member; the at least two reference indicia being spaced-apart by a respective distance;
b. capturing a reference image comprising the at least two reference indicia;
c. performing a surgical action as part of a surgical procedure that subjects the body member to strain in the region of interest of the body member;
d. capturing a further image comprising the at least two reference indicia; and
e. deriving a measure of strain within the body member from the reference image and the further image.

20. The method of claim 19, in which deriving a measure of strain within the body member from the reference image and the further image comprises comparing the change in separation of the at least two reference indica between the reference image and the further image, and calculating a measure of strain from the change in separation of the at least two reference indicia and the respective distance.
